(19) United States
(12) Patent Application Publication
(10) Pub. No.: US 2025/0236841 A1
(43) Pub. Date: Jul. 24, 2025

(54) METHODS TO ENHANCE TUMOR IMMUNOGENICITY AND COMPOSITIONS FOR AUTOLOGOUS CANCER IMMUNOTHERAPEUTIC PRODUCTS USING MODIFIED TUMOR CELLS AND MODIFIED DENDRITIC CELLS

(71) Applicant: AIVITA BIOMEDICAL, INC., IRVINE, CA (US)

(72) Inventors: Gabriel NISTOR, Laguna Niguel, CA (US); Andrew N. CORNFORTH, Mission Viejo, CA (US); Robert O. DILLMAN, Newport Beach, CA (US)

(21) Appl. No.: 18/826,085

(22) Filed: Sep. 5, 2024

Related U.S. Application Data
(63) Continuation of application No. 17/698,608, filed on Mar. 18, 2022, now abandoned.
Application No. 17/698,608 is a continuation of application No. 16/486,453, filed on Aug. 15, 2019, now abandoned.
(60) Provisional application No. 62/460,295, filed on Feb. 17, 2017.

(51) Int. Cl. C12N 5/0784; A61K 40/19; A61K 40/24; A61K 40/42; C12N 5/09

(52) U.S. Cl. CPC C12N 5/0639; A61K 40/19; A61K 40/24; A61K 40/4201; A61K 40/428; C12N 5/0693; A61K 2239/53; C12N 2501/065; C12N 2501/48

(57) ABSTRACT

The present specification provides methods for augmenting the antigenic content, especially of tumor-associated antigens (TAA), and immunogenicity of cancer cells; methods for enhancing cross-presentation in dendritic cells, compositions comprising such manipulated cells derived from single cancer patients; and methods of using those compositions as a personal immunotherapeutic product to treat the donor patient's cancer.

### CROSS REFERENCE

This application is a continuation of U.S. application Ser. No. 16/486,453, filed Aug. 15, 2019, which is a U.S. National Phase of International Application No. PCT/US2018/000023, filed Feb. 16, 2018, which claims the benefit of U.S. Provisional Application 62/460,295 filed Feb. 17, 2017. The entire contents of each of these applications are incorporated by reference herein.

### BACKGROUND

The interaction between malignant cells and the immune system includes elimination of cancer cells by the innate and adaptive immune system, especially by cytotoxic T lymphocytes (CTL) that recognize specific tumor-associated antigens (TAA), or an equilibrium between the immune system and resistant cancer cells, or the evasion of immune control that enables the escape of cancer cells and leads to eventual clinical detection of cancer. Specific immune therapies, such as the cytokine interleukin-2, can drive an existing immune response, and checkpoint inhibitors, such as anti-CTLA-4, and PD-1 and anti PD-L1, can release an anti-tumor response that was being suppressed by such inhibitors. However, a high percentage of cancer patients lack sufficient immune recognition of their malignant cells that such methods cannot successfully control or eliminate their cancer. That is, they have no evidence of CTL tumor infiltration or increased expression of PD-1 as evidence of an ongoing immune response, or of increase PDL-1 expression as evidence of a blunted immune response. Thus, there remains a need for improved methods of stimulating an anti-cancer immune response in the cancer patient.

### SUMMARY

The disclosed embodiments include autologous immunotherapy products comprising autologous dendritic cells (DC) loaded with tumor-associated antigens (TAA) from autologous cancer cells. Such products, and the pDC they comprise, are generated ex vivo, and do not encompass DC that arise in the body through natural processes or upon exposure of the body to the classes of agents disclosed herein. Nonetheless, these products and compositions, after being generated ex vivo, may be administered to a subject's body, particularly a subject in need of treatment for cancer. In various embodiments, either the DC or the cancer cells, or both, are manipulated in vitro to enhance the immunogenicity targeted to the TAA. Live cancer cells obtained from the tumor of the patient to be treated can be exposed to agents that increase the expression and/or accumulation of TAA in the tumor cells therapy increasing the quantity of TAA present, or can decrease the tolerogenicity of the cancer cells. Similarly, the cancer cells can be treated to improve their immunogenicity. The DC can be exposed to aminoglycosides which alter intracellular endosomal-lysosomal trafficking, thereby enhancing cross-presentation of exogenous antigen. The DC are exposed to lysed or whole, but non-viable, tumor cells so that the DC become loaded with, and process, TAA. The TAA-loaded DC can then be administered to the original donor cancer patient as an immunotherapeutic product. See FIG. 1.

The disclosed embodiments include methods of modifying cancer cells to improve their TAA-specific, and general, immunogenicity and methods of modifying DC to increase their level of cross presentation. Further embodiments include compositions comprising the modified DC, the modified cancer cells or lysates thereof, or both. DC are loaded with antigen by culturing DC in the presence of inactivated cancer cells or cell lysates (cancer cell material). In some embodiments, no steps are taken to remove residual cancer cell material from the DC cultures after antigen loading so compositions comprising antigen-loaded DC will include any residual cancer cell material that has not yet been taken up by the DC unless specifically stated otherwise. Autologous antigen-loaded DC are the primary component of the personalized, anticancer immunotherapeutic products described herein. Also disclosed is the use of these immunotherapeutic products in the treatment of cancer and methods of cancer treatment comprising administration of these immunotherapeutic products. Finally, specific methods of modifying DC and of modifying cancer cells to improve the immunogenicity and effectiveness of the immunotherapeutic products of which they become components are disclosed.

Cross-processing by DC can be increased by exposure to aminoglycoside antibiotics, such as gentamicin, or to Toll-like Receptor 4 (TLR-4) agonists, such as lipopolysaccharide (LPS). In some embodiments the cross-processing augmenting agent is added from the beginning of the process of differentiating monocytes into immature DC. In an aspect of these embodiments, the concentration of the cross-processing augmenting agent is relatively low. In some embodiments, the cross-processing augmenting agent is added, or the concentration increased, from the beginning of the DC maturation and antigen-loading processes. In an aspect of these embodiments, the concentration of the cross-processing augmenting agent is relatively high. In some embodiments a TLR-4 agonist, such as LPS, is used as a maturation agent.

The cancer cells can be modified by a variety of approaches relying on different mechanisms to increase TAA expression or accumulation (and thus improve the TAA-specific immunogenicity of the cancer cell material) or to increase the overall immunogenicity of the cancer cell material. In some embodiments, a method of cancer cell modification uses a single approach. In other embodiments, a method of cancer cell modification uses multiple approaches. In some embodiments, the multiple approaches rely on a single mechanism. In other embodiments the multiple approaches each rely on distinct mechanisms. In still other embodiments, some of the multiple approaches have a common mechanism, but at least one relies on a distinct mechanism. Approaches for improving accumulation of TAA include increasing protein expression by epigenetic modification, increasing protein expression by activating the PI3K/AKT/mTOR pathway, increasing protein accumulation by proteasome inhibition, increasing protein accumulation by reducing autophagy, and increasing protein accumulation by inhibiting apoptosis. Approaches for improving immunogenicity of TAA include improving accumulation of TAA, increasing general immunogenicity by removing tolerogenic molecules, and increasing general immunogenicity by increasing damage-associated molecular patterns (DAMP).

In some embodiments, the mechanism of epigenetic modification comprises demethylation of DNA or inhibition of deacetylation of histones. In some embodiments, the mechanism of activating the PI3K/AKT/mTOR pathway comprises inhibition of PTEN or the addition of growth factors or hormones. In some embodiments, the mechanism of proteasome inhibition comprises inhibition of proteasome protease activity or inhibition of ubiquitin E3 ligase. In some embodiments, the mechanism of reducing autophagy comprises inhibition of lysosomal function, for example by treatment with aminoglycoside antibiotics. In some embodiments, the mechanism of inhibiting apoptosis comprises caspase inhibition. In some embodiments, the mechanism of removing tolerogenic signals comprises depleting cholesterol and Wnt ligands. In some embodiments, the mechanism of increasing DAMP comprises reducing autophagy, for example by treatment with aminoglycoside antibiotics.

For each method of cancer cell modification, there are corresponding compositions comprising the modified cancer cells, lysates of the cancer cells, or antigen-loaded DC, the DC having been loaded with modified cancer cell material. In some embodiments the DC are modified DC. In some embodiments, particular approaches, mechanisms, or agents are specifically included. In some embodiments, particular approaches, mechanisms, or agents are specifically excluded.

In some embodiments, separate cultures of cancer cells are each modified by a different approach, mechanism, or agent and then combined together for DC antigen loading. In some embodiments, separate cultures of cancer cells are each modified by a different approach, mechanism, or agent and then used for DC antigen loading in separate DC cultures and then the antigen-loaded DC are combined in a single immunotherapeutic product. In some embodiments separate cultures of cancer cells are each modified by a different approach, mechanism, or agent and then used for DC antigen loading in separate DC cultures used to make distinct immunotherapeutic products which are separately administered to the patient. In some aspects of these embodiments, the distinct immunotherapeutic products are administered at the about the same time (within a period of minutes to 48 hours), while in other aspects the distinct immunotherapeutic products are administered at intervals of weeks or months.

### BRIEF DESCRIPTION OF DRAWINGS

FIG. 1 presents a schematic overview of a process for making an autologous immunotherapeutic anti-tumor product with improved immunogenicity using cancer cells and dendritic cells derived from the same patient.

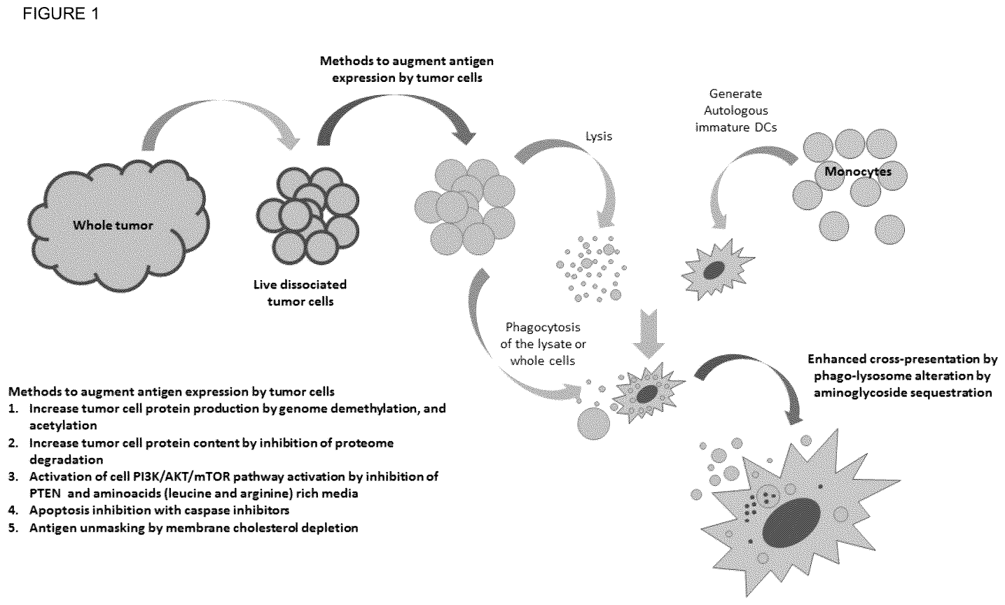

FIG. 2 presents the cell survival response to bortezomib concentration. The linear portion of the response if from about 1 μM to 5 μM.

FIG. 3 presents phase-contrast photomicrographs of ovarian tumor cell line culture 2 days after exposure to various concentrations of bortezomib.

FIG. 4 presents a phase-contrast photomicrograph demonstrating survival rescue with morphological alteration after sequential application of 20 UM of Z-VAD-fmk and 5 nM of bortezomib.

FIG. 5 presents phase contrast photomicrographs demonstrating that the combination of bortezomib and Z-VAd-fmk did not cause apparent changes in the culture morphology or survival.

FIG. 6 presents the mean fluorescence intensity of two antigens labeled with different fluors after tumor cells were exposed to various concentrations of bortezomib.

FIG. 7 presents sum of pixels of two antigens labeled with different fluors after tumor cells were exposed to various concentrations of bortezomib.

### DESCRIPTION

The immune system is able to specifically recognize and eliminate tumor cells. The potential to harness this ability in the treatment of cancer has long been recognized but success in doing so has been, at best, limited. As compared to the use of heterologous tumor cells or individual antigens or epitopes, using autologous tumor cells as immunogen offers several advantages. Cancer cells contain mutated proteins, neoantigens, which can serve as TAA, but these are frequently unique to each patient so that autologous tumor is the only reliable source. The autologous tumor will also include cancer stem cells and early progenitor cells so that TAA representing that subpopulation will be included in the immunogenic composition. Moreover, use of autologous tumor obviates the need to identify and match each individual antigen to be targeted, as would be the case in using heterologous cells or off-the-shelf immunogens. By using autologous cells as the source of antigen, the complete complement of antigens for the individual patient's cancer become included in the immunogenic composition.

However, the amount of TAA present in a tumor cell preparation may be limited either due to a low steady state level of the antigen in the tumor cells or because only some of the tumor cells express the antigen, or both. A particular example would be antigens associated with only a rare subpopulation, such as cancer stem cells. The limited amount of TAA in the tumor cell preparation can negatively impact the TAA-specific immunogenicity of the preparation. Additionally, the general immunogenicity of the cancer cells can be improved. The cancer cell can also contain tolerogenic molecules, such as Wnt ligands, the removal or depletion of which can improve the general immunogenicity of the cancer cells. The cancer cells can also contain pro-inflammatory molecules such as damage-associated molecular patterns (DAMP), the increase of which can increase the general immunogenicity of the cancer cells. DAMP can include heat-shock proteins, various nuclear and cytosolic proteins, such as HMGB-1 (High mobility group box 1 protein), membrane-bound proteins, and proteins derived from the extracellular matrix following cell injury. DAMP also include non-protein molecules such as DNA, ATP (adenosine 5′-triphosphate), uric acid, and heparin sulfate. Exposure of cancer cells to gamma interferon, many chemotherapy agents, irradiation, particular monoclonal antibodies, activated natural killer (NK) cells, cytotoxic T lymphocytes (CTL), and antibody-dependent cell-mediated cytotoxicity (ADCC) can also lead to increased DAMP signals. Thus in various embodiments, the tumor cells are exposed to one or more agents that cause an increase in protein expression, that cause a decrease in protein degradation, that promote accumulation of TAA in the tumor cells, that deplete tolerogenic molecules from the cancer cells, or that increase the cancer cells production of DAMP. Some embodiments specifically include exposure to a particular agent of class of agent. Other embodiments specifically exclude exposure to a particular agent of class of agent.

The augmenting or enhancing of protein expression not only increases the level of TAA expression in individual cells, but can also improve the uniformity with which antigens are expressed throughout the cancer cell population. The increased amount of TAA in the tumor cell preparation, by whatever mechanism, improves the likelihood that there will be sufficient material to be immunogenic. In this manner an immune response can be obtained to TAA that are naturally expressed at too low a level to be effective immunogens in the body However, the lower levels of antigen expression by the cancer cells in the body can still be sufficient to be recognized by cytotoxic T lymphocytes (CTL) and antibodies and thus lead to the destruction of the cancer cells if such an immune response can be induced in the first place.

Antigen processing of protein antigens proceeds through two paradigmatic pathways. In one, exogenous antigens are taken up by antigen presenting cells (APC), including DC, by phagocytosis and partially degraded in an endosomal compartment to produce peptides (epitopes) that become associated with class II MHC. The peptide-MHC II complexes are displayed on the surface of the APC where they are recognized by CD4⁺ T cells which, among other possibilities, can become licensed to provide T cell help to B cells, supporting the induction and maturation of an antibody response to the antigen. In the other paradigmatic pathway, endogenous protein antigens are degraded, typically by the proteosome, and the peptides (epitopes) produced become associated with class I MHC in an endosomal compartment. The peptide-MHC I complexes are displayed on the surface of the APC where they are recognized by CD8⁺ T cells which can then mature into CTL.

Antibodies can be an important part of an anti-tumor response to the extent that the TAA is expressed on the tumor cell surface. However, many TAAs are intracellular proteins and therefore not generally accessible to antibody, and rather need to be targeted by CTL. There is a variation of the paradigmatic antigen processing pathways in which exogenously encountered antigen is shunted into the endogenous antigen processing pathway, leading to antigen presentation in the context of class I MHC and the induction of a CTL response. Such cross-presentation can be increased by altering endosomal trafficking, specifically by delaying phagosome-lysosome fusion so that more phagosomal material is released into the cytosol. Such delay of phagosome-lysosome fusion can be brought about by exposure of the DC to aminoglycoside antibiotics or by activation of TLR4 through exposure of the DC to TLR4 agonists such as LPS (lipopolysaccharide), glucuronoxylomannan, and morphine-3-glucuronide. In this way the DC can more efficiently stimulate a CTL response to a broader array of TAA. The treatment does not extinguish presentation in the context of class II MHC, so humoral as well as cellular immunity is stimulated.

In some embodiments both the live cancer cells and the DC are manipulated to augment TAA expression and cross-presentation, respectively. In other embodiments, cancer cells with augmented TAA expression are used to provide antigen to unmanipulated DC. In still other embodiments the live cancer cells that have not been manipulated to augment TAA expression are used to provide antigen to DC that have been manipulated to augment cross-presentation. However, the combined effect of augmented antigen availability for uptake by DC and enhanced cross-presentation by the DC is expected to synergistically improve the immunogenicily of the manipulated, antigen-loaded DC as a cellular immunotherapeutic product. Thus in various embodiments, an immunotherapeutic product is made using modified cancer cells (or lysates thereof), modified DC, or both.

In some embodiments the patient is a human. In other embodiments the patient is a non-human mammal, for example a canine, feline, or equine patient. In some embodiments the non-human mammal is not a rodent.

Isolation of Live Cancer Cells from Tumors

Live tumor cells are removed from the body of the cancer patient during tumor removal or de-bulking surgery. In some instances surgery will entail the removal of whole tumor(s). In some instances it will entail removal of whole organs or substantial portions thereof. When diseased and normal tissue are both present in the removed tissue, tumor tissue is dissected away from the normal tissue. In other instances surgery entails a biopsy, including but not limited to, punch or needle biopsies. For solid tumors, the tissue is minced and dissociated by enzymatic digestion. In the instance of a leukemia, live cancer cells can be recovered from blood, for example by density gradient sedimentation of whole blood or by leukapheresis. In the instance of an ascites tumor, live tumors cells can be recovered by draining the ascites fluid followed by sedimentation. The number of live cancer cells recovered will vary with tumor size and the particular method of recovery, but generally more are preferred, especially for tumors that may proliferate poorly in culture. Thus in various embodiments, on the order of 10⁵ to 10⁷ to 10⁹ or more live cancer cells are recovered. Following separation from digested extracellular matrix and other debris, the live cells are transferred into a rich cell culture medium for expansion and/or exposed to one or more agents to augment the expression and accumulation of TAA.

In some embodiments, the tumor or cancer cells are from any malignant neoplasia. Some embodiments will specifically include, and other embodiments will specifically exclude, a particular class(es) or type(s) of cancer. They can be classified as forming solid tumors or as comprising cells suspended in a bodily fluid. They can be classified according to tissue of origin, such as brain, head & neck, esophagus, lung, liver, pancreas, kidney, stomach, colon, prostate, breast, uterus, cervix, ovary, skin, bone, hematologic, ocular, or retinal. They can be classified as a particular type, for example, melanoma, non-small cell lung cancer, glioblastoma, renal cell carcinoma, etc. They can be further subdivided according to biomarker expression such as triple negative breast cancer, hormone resistant prostate cancer, PD-L1 positive (or negative) lung cancer, etc. They can also be subdivided according to disease progression: non-invasive, invasive, metastatic; stage 0, 1, 2, 3, or 4; and various scales related to specific cancers. Cancers can also be classified according phenotypically significant mutations that they carry, for example mutations in p53 or B-Raf.

### Augmented and Enhanced Antigen Expression and Immunogenicity of Isolated Cancer Cells

A variety of agents are available which by one mechanism or another can increase the accumulation or exposure of TAA or reduce the tolerogenicity of the tumor cells. These include increasing protein expression, reducing protein degradation, inhibiting apoptosis, and depleting cholesterol and Wnt ligands from the cell membrane. In some embodiments, the isolated live cancer cells will be cultured from 24 hours to 4 weeks. In other embodiments, the period of culture will extend from 4 to 6 weeks, 4 to 8 weeks, or longer. In still other embodiments, the period of culture will be short, lasting only a few hours, for example 2, 3, 4, 5, 6, or more hours, but not to exceed 24 hours. In some embodiments, the period of culture will be divided into two phases, a proliferation phase, to increase the number of cancer cells available, followed by an augmentation phase, in which the cancer cells are modified to improve their overall immunogenicity. In other embodiments, the proliferation phase and the augmentation phase will coincide or there will be no proliferation phase. Depending on the augmenting agent and its mechanism of action, in some embodiments, the augmenting agent will be present throughout the augmentation phase. In other embodiments the augmentation agent will be present only for the final several hours of culture or only the final day of the augmentation phase, or will be present throughout the augmentation phase but the agent's concentration will be increased in this final period. In still other embodiments, the augmentation agent will be present only for the initial several hours of culture or only the initial day of the augmentation phase, or the agent's concentration will be decreased after this initial period but maintained at the lower concentration throughout the remainder of the augmentation phase. In some embodiments, the cancer cells will be exposed to the agent(s) during just one of the augmentation procedures described herein below. In other embodiments, the cancer cells will be exposed to the agents for multiple augmentation procedures, for example 2, 3, 4, or more of the augmentation procedures described herein below. In some embodiments, these modified cancer cells will be exposed to autologous DC immediately following the augmentation phase. In other embodiments, these modified cancer cells will be cryopreserved for exposure to autologous DC at a later time.

### Epigenetic Modification

Protein expression levels can be increased by epigenetic modification of cancer cells. In comparison to physiologically normal cells, cancer cells possess epigenetic modifications that reduce the expression of various proteins. Thus, by reversing the acquired epigenetic modifications of the cancer cells, expression of down-regulated TAA can be restored.

Transcriptional activation is associated with the acetylation of lysine residues in histone tails. One epigenetic mechanism by which protein expression can be down-regulated is de-acetylation of lysine residues in histone tails. Exposure to histone deacetylase inhibitors (HDI) can be used to increase mRNA transcription with a concomitant increase in protein expression and the antigenic content of cancer cells. Examples of HDI include hydroxamic acids (or hydroxamates), such as trichostatin A, cyclic tetrapeptides (such as trapoxin B), and the depsipeptides, benzamides, electrophilic ketones, and aliphatic acid compounds such as phenylbutyrate and valproic acid. Second-generation HDIs include the hydroxamic acids vorinostat (SAHA), belinostat (PXD101), LAQ824, and panobinostat (LBH589); and the benzamides: entinostat (MS-275), C1994, and mocetinostat (MGCD0103). Additionally, class Ill histone deacetylases are dependent on NAD* and are, therefore, inhibited by nicotinamide, as well as derivatives of NAD, such as dihydrocoumarin, naphthopyranone, and 2-hydroxynaphthaldehydes. Thus these agents can be used to increase the level of transcription activation and expression of TAA.

Promoters for protein coding genes typically have an increased frequency of CG dinucleotides, which are referred to as CpG islands. These CG dinucleotides can be methylated and hypermethylation of these dinucleotides in the CpG islands can result in transcriptional silencing. Such hypermethylation is a stable epigenetic change that can be inherited by daughter cells following mitosis. While such silencing plays a role in normal physiology, for example in regulating gene dosage, abnormal hypermethylation is commonly seen in cancer. Demethylating agents can be used to turn back on the expression of silenced genes including germ-line or tumor-specific genes that may be expressed by cancer cells.

Demethylation agents such as 5-azacytidine (azacitidine, 5-aza-CR; Vidaza®, Celgene Corp., Summit, NJ, USA) and 5-aza-2′-deoxycytidine (decitabine, 5-aza-CdR; Dacogen®, SuperGen, Inc., Dublin, CA, USA) were previously used as anticancer agents-though operating through a different mechanism. At high concentrations these drugs disrupt normal polynucleotide physiology to an extent that is cytotoxic. Azacitidine, which incorporates preferentially into RNA, disrupts protein synthesis, but decitabine incorporates only into DNA and at low concentrations inactivates DNA methyltransferases and disrupts the heritability of CpG methylation patterns. Thus these demethylating agents can be used increase expression of TAA and preferred embodiments use decitabine as a demethylating agent.

### Inhibition of Proteome Degradation

Proteasome inhibitors are well known therapeutic agents used to disrupt the tumor cell protein turnover triggering cell death by caspase activation. In normal cells, the proteasome regulates protein expression and function by degradation of ubiquitylated proteins, and also cleanses the cell of abnormal or misfolded proteins.

The proteosome is the major neutral proteolytic apparatus of the cell and plays a primary role in normal protein turnover, as well as in the degradation of damaged, misfolded, and abnormal proteins in the cytosol and nucleus, especially those proteins that have become ubiquitinated. Long-term blockage of protein breakdown will lead to cell death, but initially should lead to an accumulation of proteins otherwise destined for degradation. Inhibitors of the proteasome and enzymes of unbiqutination pathway, especially the E3 ligases, can be used to block protein degradation.

Due to transcriptional and translational failures, genomic mutations or diverse stress conditions like oxidation or heat, misfolded proteins are produced in every compartment of the cell. Misfolded proteins are targeted to proteolytic pathways, most prominently the ubiquitin-proteasome system and the autophagic vacuolar (lysosomal) system. Thus, inhibiting the ubiquitin, proteasome system we cause an accumulation of misfolded or mutated (neoantigenic) proteins that may have an antigenic value. As a secondary outcome, the increased lysosomal processing may lead to increased MHC presentation to immune system.

While multiple mechanisms are likely to be involved, it is believed that proteasome inhibition prevents degradation of pro-apoptotic factors, thereby triggering programmed cell death in neoplastic cells. It was also shown that proteasome inhibition alters the balance of intracellular peptides after short administration at relatively high doses (50-500 nM).

A variety of non-peptidic and peptidic, reversible and irreversible inhibitors of the 20S proteasome have been identified that can enter mammalian cells and inhibit degradation of proteins by the ubiquitin-proteasome pathway. The first non-peptidic proteasome inhibitor discovered was the natural product lactacystin. Other proteasome inhibitors include disulfiram, epigallocatechin-3-gallate, marizomib (salinosporamide A), oprozomib (ONX-0912), delanzomib (CEP-18770), epoxomicin a naturally occurring selective inhibitor, beta-hydroxy beta-methylbutyrate (HMB), bortezomib; carfilzomib and ixazomib. E3 ligase inhibitors include nutlin-3, JNJ-26854165 (serdemetan), NVP-CGM097, NSC 207895, N-(4-butyl-2-methylphenyl) acetamide (SKP2 E3 ligase inhibitor II), 5-(3-dimethylaminopropylamino)-3,10-dimethyl-10H-pyrimido[4,5-b]quinoline-2,4-dione (Hdm2 E3 ligase inhibitor II), and 9H-indeno[1,2-e][1,2,5]oxadiazolo[3,4-b]pyrazin-9-one (SMER 3). Thus these agents can be used to cause accumulation of TAA in cancer cells.

The in vivo use of bortezomib was shown to dramatically impair the ability of native human blood DCs to regulate innate and adaptive anti-tumor Immunity which has implications for the design of therapeutic strategies combining DC vaccination and bortezomib treatment. Use of proteasome inhibitors also results in the accumulation of caspase cascade components that normally are degraded by proteasome, leading to cell death by apoptosis.

In some embodiments, ex vivo treatment of cancer cells with a proteasome inhibitor, such as bortezomib, is utilized to circumvent the impairment and death of DC arising from in vivo exposure. By utilizing ex vivo treatment exposure of the DC to the proteasome inhibitor can be avoided or reduced. Additionally, the blockade of the caspases with a pan-caspase inhibitor (i.e. Z-VAD-fmk) will prevent the initiation of apoptosis, whether of the tumor cell or the DC, either of which could be counterproductive.

By simultaneous or sequential use of a caspase inhibitor (e.g., Z-VAD-fmk for broad spectrum caspases) and a proteasome inhibitor (e.g., bortezomib) the accumulation of variety of peptides and proteins including tumor neoantigens can be expected, thereby increasing antigenic load to trigger a better immune response. Furthermore, the in vitro use of the proteasome inhibitor with tumor cells avoids exposing DC to the proteasome inhibitor and thus does not interfere with the antigen presentation and antitumor immune activation functions of DC.

### Reduction of Autophagy

Aminoglycoside antibiotics can be toxic to mammalian cells due to selective accumulation in lysosomes and inhibition of lysosomal enzymes. In vitro treatment of tumor cells with gentamicin in the absence of iron ions can decrease the lysosomal processing and increase TAA accumulation along with enhanced DAMP signaling, favorable for phagocytosis by DC. This effect is more prominent following stresses that increase autophagy, such as irradiation or starvation. Thus aminoglycoside antibiotics such as gentamicin can be used to reduce or delay autophagy and thereby increase the protein “junk” in the cell leading to increased DAMP signaling. In some embodiments the cancer cells will be stressed and then exposed to 50-150 μg/ml of gentamicin. The increased DAMP signaling enhances the general immunogenicity of the cancer cells in addition to the accumulation of protein including TAA.

### Activation of the PI3K/AKT/mTOR Pathway

The PI3K/Akt/mTOR pathway is a complex signaling pathway involved in many cellular processes including cell proliferation and survival, cell growth and differentiation, insulin action, and the control of protein synthesis and autophagy, as well as having a central mle in maintaining the malignant state in many cancers. Mechanisms for pathway activation include inhibition of tumor suppressor PTEN function, amplification of phosphatidylinositol-4,5-bisphosphate 3-kinase (PI3K), amplification or mutation of Akt, and amplification of growth factor receptors. One of the downstream effects of pathway activation is activation of mTOR a master regulator of protein translation. mTOR exists in two complexes: the TORC1 complex and the TORC2 complex. In the TORC1 complex, mTOR signals to its downstream effectors S6 kinase/ribosomal protein S6 and 4EBP-1/eIF-4E to control protein translation. Although mTOR is generally considered a downstream substrate of Akt, mTOR can also provide a positive feedback through TORC2 complexes to the pathway.

Both mTORC1 and mTORC2 respond to hormones and growth factors. mTORC1 in particular also appears to be acutely regulated by nutrients, such as amino acids and glucose. The presence of amino acids in the cell culture media, specifically of leucine and arginine in higher quantity, enables increasing cell growth through increased ribosome biogenesis and protein biosynthesis, and suppression of autophagy. Proliferation in human hepatoma cell lines has been shown to be dependent on the concentration of leucine in vitro, with a significant reduction in proliferation rates in 0.05 mM leucine-containing medium compared to 0.2 mM. In various embodiments, the molar ratio of leucine or arginine to alanine is at least 10:1 or more, for example 25:1, 50:1, or 100:1. Thus higher than standard levels of leucine and/or arginine in the culture medium can increase proliferation and protein expression (including TAA expression) in the cultured cancer cells.

PTEN (Phosphatase and Tensin Homolog deleted on Chromosome 10) is a phosphatidylinositol-3,4,5-trisphosphate 3-phosphatase that converts phosphatidylinositol-3,4,5-trisphosphate to phosphatidylinositol-4,5-disphosphate thus opposing PKB/Akt activation by PI3K. PTEN is a 50 kD cytosolic enzyme that interacts transiently with the plasma membrane to metabolize its lipid substrate. Loss of function through several distinct mechanisms has been observed at high frequency in many tumor types. PTEN activity suppression has potent effects in many cell lineages on cell proliferation, growth, survival and associated changes in metabolism. Vanadium compounds, such as sodium orthovanadate, have long been recognized as phosphatase inhibitors. Peroxovanadium compounds such as bisperoxovanadium 1,10 phenanthroline (bpV(phen)) and bisperoxovanadium 5-hydroxypiridine-2-carboxyl(bpV(HOpic)) exhibit increased biological potency and have greater target selectivity than the simple vanadate compounds. N-(9,10-dioxo-9,10-dihydrophenanthren-2-yl)pivalamide (SF1670) is also a potent and specific inhibitor of PTEN. In some embodiments, PTEN inhibitors are used in combination with agents acting through the PI3K pathway. Such factors include known growth factors (for example, fibroblast growth factor (FGF), epidermal growth factor (EGF), VGF nerve growth factor inducible (VGF), hepatocyte growth factor (HGF), insulin-like growth factor (IGF), etc.), hormones, integrins (laminins), and other receptor-mediated signaling factors. Thus these factors can be used to increase the level of cellular activation-including protein synthesis—in order to increase the level of TAA expression in the cultured cancer cells. A preferred combination of factors added in the cell culture media is insulin, thyroid hormone, basic FGF and EGF. Thus inhibitors of PTEN, especially when used in combination with growth factors enable the accelerated growth and proliferation of the cancer cells as well as increased protein synthesis.

### Inhibition of Apoptosis

Apoptosis is a regulated process of programmed cell death initiated by various stresses and biochemical signals. It contrasts with necrosis which arises from traumatic injury to cells. The process is regulated by a family of enzymes called caspases, cytosolic aspartate-specific cysteine proteases. They are responsible for the initiation and execution of apoptotic program. The caspases are expressed as latent zymogens and are activated by an autoproteolytic mechanism or by processing by other proteases (frequently other caspases). Human caspases can be subdivided into three functional groups: cytokine activation (caspase −1, −4, −5, and −13), apoptosis initiation (caspase −2, −8, −9, - and −10), and apoptosis execution (caspase −3, −6, and −7). Caspases respond to a variety of stimuli, including APAF1, CFLAR/FLIP, NOL3/ARC, and members of the inhibitor of apoptosis (IAP) family such as BIRC1/NAIP, BIRC2/cIAP-1, BIRC3/cIAP-2, BIRC4/XIAP, BIRC5/Survivin, and BIRC7/Livin. IAP activity is modulated by DIABLO/SMAC or PRSS25/HTRA2/Omi. The in vitro exposure to broad spectrum caspase inhibitors could prevent cell death and allow rapid tumor cell expansion with the accumulation of TAAs.

Non-limiting examples of cell-permeable and reversible or irreversible, peptide or non-peptidic caspase inhibitors from natural or synthetic sources are included in Table 1. These agents can be used to increase the number and proportion of live cancer cells in the culture and allow the accumulation of TAA.

TABLE 1
Caspase inhibitors

| Compound | Source | Target |
|---|---|---|
| CrmA | Cowpox virus | Caspase 1, 8, 10 |
| P35 | Baculovirus | Broad spectrum, caspase 1, 3, |
| | | 6, 8, 7 and 10 |
| P49 | Baculovirus | Broad spectrum, like p35, |
| | | including DRONC and caspase 9 |
| DIAP1 | Drosophila | DRONC, DrICE |
| XIAP | Mammalian | Caspase 3, 7, 9 |
| Q-VD-Oph | Synthetic peptidic | Caspases 1, 3, 8 & 9 |
| DEVD-CHO | Synthetic peptidic | Caspase 3, 7 |
| Z-VAD-fmk | Synthetic peptidic | All caspases, caspase 2 |
| | | very weakly |
| IDN-6556 | Synthetic non-peptide | Broad spectrum, irreversible |
| Pralnacasan | Synthetic non-peptide | Caspase 1 |
| M867 | Synthetic non-peptide | Reversible, selective caspase |
| | | inhibitor |

### Depletion of DC-Inhibitory Signaling Molecules

A common mode of cell-to-cell communication is the secretion of signaling molecules that are then received by neighboring cells. One such signaling system is mediated by the Wnt ligands, a large family of hydrophobic glycoproteins that are lipid modified. Indeed it is the lipid modification that provides their hydrophobic character as the primary sequence appears relatively hydrophilic. Reception of Wnt ligands at a cell's surface initiates an intracellular signaling cascade leading to changes in gene transcription. In DC, Wnt signaling leads to activation of β-catenin, a critical step in promoting tolerance and limiting inflammation.

Aberrant regulation of Wnt signaling is common across many tumor types. The epigenetic and genetic alterations related to the malignant state result in elevated Wnt pathway activity. More recently, it has become apparent that Wnt signaling levels identify stem-like tumor cells that are responsible for fueling tumor growth. In tumors, the accumulation of Wnt signaling is partially responsible for immune escape by the modulation of the antigen presenting cells with excessive Wnt signaling. Moreover, one of the most desirable targets of cancer immunotherapy, the cancer stem cell, appears to be particularly tolerogenic.

Wnt ligands are preferentially found in lipid rafts, detergent-resistant portions of the cell's plasma membrane rich in cholesterol, gangliosides, and sphingolipids. Depletion of membrane cholesterol disrupts integrity of lipid rafts and concurrently depletes Wnt. Among various cholesterol-depleting agents available, methyl-β-cyclodextrin (MCD), a highly water soluble cyclic heptasaccharide consisting of β-glucopyranose unit, is the most effective agent for depletion of cholesterol, along with other lipid modified membrane components including Wnt ligands, from the cells. In the case of Wnt-rich tumors, the capture and removal of Wnt ligands from tumor cells prior to exposure to DC will prevent programming the DC to a tolerogenic state and thus improve the immunogenicity of the cancer cell preparation. Thus treatment with cholesterol-depleting agents such as MCD will also deplete Wnt ligands leading to improved immunogenicity of the tumor cells and especially of cancer stem cells.

### Augmentation of Antigen Processing

Cytolytic immune responses are primarily mediated by CD8⁺ T cells. To stimulate such a response, DC need to present antigen in the context of class I MHC which is predominantly loaded with endogenously expressed antigen. Phagocytosed material is predominantly presented in the context of class II MHC, but there is a process, called cross-presentation, that leads to the presentation of phagocytosed antigen in the context of class I MHC. After uptake, phagosomes undergo sequential fusion and fission events, first with endosomal and then lysosomal compartments leading to degradation of the phagosome content, a process referred to as “phagosome maturation”. DCs have developed a specialized phagocytic pathway which allows optimal conditions for cross-presentation. These specializations include a mildly degradative phagosomal environment, export of antigen to the cytosol for proteasome-mediated degradation, and effective loading of the generated peptides in the endoplasmic reticulum (ER) or in phagosomes. The normal phagosome lysosome fusion leads to the degradation of the phagosome content. Delays in the fusion process results in enhanced cytosolic export of the phagosome content. Thus, an intended delay in phagosome processing of the tumor cells can result in increased cross-presentation of TAAs and enhanced cytotoxic immune response.

Aminoglycoside antibiotics accumulate in lysosomes and inhibit lysosomal enzymes leading to a build of autophagic material. The accumulation of the material triggers cellular stress responses including production of reactive oxygen species (ROS). In the presence of ROS and lysosomal iron, the lysosomal membrane is permeated and the content is released into cytosol causing apoptosis and cell death. However the toxicity for DC can be reduced by the absence of iron in the culture medium. Thus low concentrations of an aminoglycoside antibiotic such as gentamicin leads to an enhanced level of cross presentation. TLR4 agonists also mediate a delay in phagosome-lysosome fusion. Thus in some embodiments the aminoglycoside antibiotic is supplemented with a TLR4 agonist such as LPS, glucuronoxylomannan, or morphine-3-glucuronide.

### Isolation of Dendritic Cells

DC for use in the embodiments described herein can be obtained by differentiation of monocytes isolated from the blood of the same patient as the tumor cells are isolated from. Techniques for the differentiation of DC from monocytes are well established in the art. Briefly, in a typical protocol, peripheral blood mononuclear cells (PBMC) are isolated from whole blood by density gradient centrifugation. The PBMC are plated. Monocytes are adherent and non-adhierent cells are washed away after 1-24 hours. Alternatively, immuno-magnetic beads may be used to isolate the monocytes from the PBMC. The monocytes are cultured in the presence of GM-CSF and IL-4 for 5-8 days to differentiate into immature DC. At this point, the immature DC are loosely adherent and can be harvested by gentle pipetting. The immature DC are then cultured another ˜2 days in the presence of maturation factors, typically a TLR-4 agonist such as LPS. Alternatively, a monocyte maturation cocktail, for example comprising TNFα, IL-6, IL-1β, and PGE2, can be used. Typically, maturation and antigen loading are carried out simultaneously. The procedure can be carried out with freshly isolated or cryopreserved PBMC.

### Harvest and Inactivation of Cancer Cells Following TAA Augmentation

The cancer cell cultures are harvested by enzyme digestion (for example, with trypsin TrypLE, collagenase, or dispase) or mechanical scraping after being subjected to one or more procedures to augment expression or accumulation of TAA or enhance immunogenicity including, but not limited to, those herein described. The cells are collected and washed free of culture medium and enzyme solution by repeated cycles of sedimentation in a neutral buffer (for example, phosphate buffer, saline, Hanks balanced salt solution, Ringer's, or the like). Total protein can be determined by the biuret method or spectrophotometric methods using dyes (Bradford, 3′,3″,5′,5″-tetrabromophenolphthalein ethyl ester-TBPEE, or erythrosin-B).

As these cancer cells will be used in preparing an immunotherapeutic product that will be administered to the patient/donor, it is important that they be inactivated (made incapable of undergoing cell division) so as to ensure that viable malignant cells are not re-administered to the patient. One method of inactivation is gamma irradiation by exposure to a radioactive source (for example, Cs-137, Co-60) to a total cumulative dose of 10-100 Gy (1,000-10,000 Rad). Alternatively, exposure to X-rays or UV irradiation can be used for the same purpose. Irradiated whole cells can then be combined with DC for antigen loading.

Cell lysis can be used for inactivation instead of, or in addition to, irradiation. Lysis can be obtained by exposure to repeated freeze-thaw cycles in isotonic or hypotonic solutions and the absence of cryoprotectants. A mechanical lysis can be also produced by exposure to high intensity ultrasound using a sonicator. Bath or probe type sonicators are both acceptable, though particular care must be exercised with the latter to avoid cross-contamination of samples. An osmotic lysis can be obtained by exposure of the cells to hypotonic buffer. Other methods of lysis consistent with current Good Manufacturing Practices can also be used. The lysate can then be combined with DC for antigen loading. Cancer cell lysates and inactivated whole cancer cells shall be referred to collectively as cancer cell material.

Various quality control methods to assess whether inactivation has been complete are available. One method is dye exclusion, in which live cells exclude the dye but inactivated cells become stained. Appropriate dyes include trypan blue, which can be used for assessment by light microscopy or in an automated fashion using a cell counting machine (for example the Vi-CELL™; Beckman Coulter), and propidium iodide or 7-Aminoactinomycin D (7-AAD), which can be use for assessment by fluorescence microscopy or flow cytometry. Viability can also be assessed according to particle size analysis using cell counter machines or flow cytometers. Finally, there are a variety of proliferation assays that can be used to detect viable cells. These include proliferation assays that rely on the incorporation of radio-labeled nucleotides into DNA and assays relying on chromogenic products such as the formazan dyes formed by the reduction of a corresponding tetrazolium salt as in the MTT and MTS assays.

In some instances, the patient may not be ready to receive the immunotherapeutic product. For example the immunotherapy regimen may call for multiple rounds of administration on a particular schedule, yet the time for the next administration has not arrived. In such instances, the inactivated cells or cell lysates may be frozen for future use. In the instance of whole inactivated cells, a cryoprotectant such as DMSO, glycerol, trehalose, sucrose, or the like is used and the cells stored at about −135° C. to about −196° C., such as in an LN2 freezer. Lysates may be stored at −20° C. or lower. In some embodiments the cancer cells would be maintained in culture throughout the treatment period or some substantial portion thereof, allowing multiple cycles of augmentation phase culture so that a fresh harvest may be made for each scheduled administration. In other embodiments, a single augmentation phase and harvest provides cancer cell material for multiple, even all, administrations.

### Antigen Loading of the DC

The inactivated augmented cancer cells or cell lysate (cancer cell material) is added to cultures of immature DC along with a maturation factor such a LPS. The DC are cultured for a further period of time to allow uptake of antigen and antigen processing to occur. In various embodiments this antigen processing phase continues for 4 to 36 hours. In preferred embodiments the DC have been treated with an aminoglycoside antibiotic, in which case such treatment is continued during the processing phase. At the end of the antigen processing phase, aliquots of the DC are frozen in liquid nitrogen in the presence of cryoprotectant until needed. In some embodiments, the antigen loaded DC are purified away from the cancer cells or cell lysates prior to administration to the patient. In other embodiments the mixture is administered to the patient. To purify the DC away from a lysate, simple sedimentation and resuspension washes can be employed. Immuno-affinity methods, for example immuno-magnetic beads, can be used to remove whole cancer cells remaining after the antigen processing phase. In other embodiments, the antigen preparation and DC are not separated; the mixture is used in the immunogenic composition.

### Immunization

The TAA-loaded DC (the combined cancer cell material and DC) are administered to the patient/donor. Administration can be by injection or infusion by any medically appropriate route of injection including for example intravenous, subcutaneous, intramuscular, intradermal, intralymphatic (that is, into an afferent lymphatic vessel), intranodal (for example, into an inguinal or axial lymph node). The TAA-loaded DC can be used in a stand-alone immunotherapy or in combination with an immune checkpoint inhibitor such as an antibody to CTLA4 (for example ipilimumab), PD-1 (for example, pembrolizumab or nivolumab), and/or PD-L1 (for example atezolizumab).

In some embodiments, doses are administered at one week intervals for the first month and then monthly for a total of 8 doses. Other embodiments comprise only a single dose. Other embodiments continue periodic administration until no disease is detected or there is frank progression of the disease. In still other embodiments, the immunotherapeutic product is administered as a continuous infusion, for example, over hours, days, weeks, or months. In some embodiments new batches of product are produced from new metastases if, and when, they appear. The number of DC administered can vary widely, such as from about 1 to about 20 million DC per administration, for example 10 million. However, more or fewer cells may also be used. For example, in some embodiments using multiple bolus injections, the number of DC per injection can be in the lower end of the above range or even below it. For example, in some embodiments using infusion over an extended period of time, the total number of DC administered can be toward the high end of the above range or exceed it. In some embodiments the number of DC. administered will be limited by the amount of tumor tissue or DC that can be obtained from the patient.

### Logistics

The expertise and infrastructure to carry out the procedures described herein may not be available in every hospital, clinic, out-patient surgery center, and the like (collectively, patient treatment sites) where a cancer patient may receive treatment. In some embodiments, a central facility with the necessary infrastructure and trained personnel receives tumor tissue and blood of a patient in need of treatment from a patient treatment site. The central facility carries out procedures to modify cancer cells and/or DC as described herein to generate a personalized anti-cancer immunotherapeutic product comprising autologous DC loaded with autologous TAA, and sends the immunotherapeutic product to a patient treatment site where the immunotherapeutic product can be administered to the patient. In some embodiments, the central facility carries out particular modifications of the cancer cells and/or DC according to instructions from the patient's doctor that in the doctor's judgement will be of particular benefit to the patient. In some embodiments the immunotherapeutic product is administered to the patient according to guidance provided by the central facility (such as the time frame within which administration should occur, dosage, frequency of administration, rate of infusion if infused, or how the immunotherapeutic product should be stored between receipt and administration) and the patient benefits thereby (and the doctor and/or patient treatment site receives the benefit of being able to treat the patient). The patient treatment site where the tumor tissue is removed from the patient and the patient treatment site where the immunotherapeutic product is administered can be the same or different. By “central facility” it is meant that the facility serves multiple patient treatment sites. The central facility can be co-located in the same building or campus as one of the patient treatment sites or it may be separately located from all patient treatment sites.

In some instances the patient receives a benefit in the form of an improvement in their cancer (that is, the cancer ceases progression, regresses, or goes into remission, or secondary symptoms improve, or they avoid the adverse side-effects of other treatments). In aspects of these embodiments the doctor, other healthcare professionals, healthcare facilities (such as the patient treatment sites), health maintenance organizations, and/or central facility are acting at the behest of the patient. In other aspects the patient's benefit is contingent on consenting to the tissue donations and receiving administration of the immunotherapeutic product according to the instruction and direction of their doctor, other healthcare professionals, healthcare facilities (such as the patient treatment sites), health maintenance organizations, and/or central facility, or on arranging for payment for the various necessary steps of the methods to be carried out. In other instances the doctor, other healthcare professionals, healthcare facilities (such as the patient treatment sites), health maintenance organizations, and/or central facility receives a benefit to their reputation or business by obtaining positive outcomes for their patients, or by being paid to carry out one or more necessary steps of the method, and the patient or other parties in the rest of the chain of the doctor, other healthcare professionals, healthcare facilities (such as the patient treatment sites), health maintenance organizations, and/or central facility are acting at their behest. In other aspects the doctor's, other healthcare professionals′, healthcare facilities' (such as the patient treatment sites′), health maintenance organizations′, and/or central facility's benefit is contingent on upon the instruction or direction of the patient or other party in the rest of the chain of the doctor, other healthcare professionals, healthcare facilities (such as the patient treatment sites), health maintenance organizations, and/or central facility.

The cryopreserved immunotherapeutic product (i.e., the cryopreserved, antigen-loaded DC) is stored at the central facility until the patient is ready for a dose. A single dose is then shipped to the patient treatment site where it is thawed and administered to the patient without further processing or manipulation. If the patient treatment site and central facility are co-located and the patient is ready to receive the immunotherapeutic product when the antigen processing phase will end, it is not required to cryopreserve the immunotherapeutic product, but instead it can be promptly administered to the patient.

### List of Particular Embodiments

The following listing of embodiments is illustrative of the variety of embodiments with respect to breadth, combinations and sub-combinations, class of invention, etc., elucidated herein, but is not intended to be an exhaustive enumeration of all embodiments finding support herein.

Embodiment 1. A composition comprising cancer cells and dendritic cells (DC) from a same cancer patient, wherein the cancer cells or the DC, or both, have been modified ex vivo to improve the accumulation, or immunogenicity, of tumor-associated antigens (TAA) expressed by the patient's cancer.

Embodiment 2. A composition comprising DC from a cancer patient, wherein the DC have been loaded with cancer cell material isolated from tumor removed from the cancer patient, wherein the cancer cells or the DC, or both, have been modified to improve the accumulation, or immunogenicity, of TAA expressed by the patient's cancer.

Embodiment 3. The composition of embodiments 1 or 2 wherein the modification to improve the accumulation of TAA comprises increasing protein expression by epigenetic modification.

Embodiment 4. The composition of any one of embodiments 1-3 wherein the modification to improve the accumulation of TAA comprises increasing protein expression by activating the PI3K/AKT/mTOR pathway.

Embodiment 5. The composition of any one of embodiments 1-4 wherein the modification to improve the accumulation of TAA comprises increasing protein accumulation by proteasome inhibition.

Embodiment 6. The composition of any one of embodiments 1-5 wherein the modification to improve the accumulation of TAA comprises increasing protein accumulation by reducing autophagy.

Embodiment 7. The composition of any one of embodiments 1-6 wherein the modification to improve the accumulation of TAA comprises increasing protein accumulation by inhibiting apoptosis.

Embodiment 8. The composition of any one of embodiments 1-7 wherein the modification to improve immunogenicity of TAA comprises removing tolerogenic molecules.

Embodiment 9. The composition of any one of embodiments 1-8 wherein the modification to improve immunogenicity of TAA comprises increasing general immunogenicity by increasing damage-associated molecular patterns (DAMP).

Embodiment 10. The composition of any one of embodiments 1-9, wherein the DC have been modified to have an increased level of cross-presentation.

Embodiment 11. The composition of embodiment 10, wherein the DC have been modified by exposure to an aminoglycoside antibiotic.

Embodiment 12. The composition of embodiment 11, wherein the aminoglycoside antibiotic comprises gentamicin.

Embodiment 13. The composition of any one of embodiments 10-12, wherein the DC have been modified by exposure to a toll-like receptor 4 agonist.

Embodiment 14. The composition of any one of embodiments 1-13, wherein the cancer cells have been modified to express or accumulate an increased amount of TAAs.

Embodiment 15. The composition of embodiment 14, wherein the cancer cells have been modified by exposure to a genome demethylation agent.

Embodiment 16. The composition of embodiment 15, wherein the genome demethylation agent comprises decitabine.

Embodiment 17. The composition of embodiment 14, wherein the cancer cells have been modified by exposure to a histone acetylation-promoting agent.

Embodiment 18. The composition of embodiment 17, wherein the histone acetylation-promoting agent comprises a histone deacetylatase inhibitor.

Embodiment 19. The composition of claim 18, wherein the histone deacetylase inhibitor comprises valproic acid.

Embodiment 20. The composition of embodiment 14, wherein the cancer cells have been modified by exposure to a proteasome inhibitor.

Embodiment 21. The composition of embodiment 20, wherein the proteasome inhibitor comprises lactacystin, epoxomicin, beta-hydroxy-beta-methylbutyrate, or any combination thereof.

Embodiment 22. The composition of embodiment 14, wherein the cancer cells have been modified by exposure to an E3 ligase inhibitor.

Embodiment 23. The composition of embodiment 14, wherein the cancer cells have been modified by exposure to a PI3K/AKT/mTOR pathway activator.

Embodiment 24. The composition of embodiment 23, wherein the PI3K/AKT/mTOR pathway activator comprises supranormal concentrations of leucine or arginine, or both, in the cell culture medium.

Embodiment 25. The composition of embodiment 23 or 24, wherein the PI3K/AKT/mTOR pathway activator comprises a PTEN inhibitor.

Embodiment 26. The composition of embodiment 25, wherein the PTEN inhibitor comprises bisperoxovanadium-1,10-phenanthroline (bpV(phen), bisperoxovanadium-5-hydroxypiridine-2-carboxyl (bpV(HOpic), bisperoxo-(bipyridine)-oxovanadate bpV(bipy), or any combination thereof.

Embodiment 27. The composition of embodiment 25 or 26, wherein the PTEN inhibitor is used in conjunction with one or more hormones or growth factors.

Embodiment 28. The composition of embodiment 27, wherein the one or more hormones or growth factors comprise insulin, thyroid hormone, basic FGF, EGF, or a combination thereof.

Embodiment 29. The composition of embodiment 14, wherein the cancer cells have been modified by exposure to an inhibitor of apoptosis.

Embodiment 30. The composition of embodiment 29, wherein the inhibitor of apoptosis is a caspase inhibitor.

Embodiment 31. The composition of embodiment 30, wherein the caspase inhibitor comprises Z-VAD-fmk.

Embodiment 32. The composition of any one of embodiments 1-13, wherein the cancer cells have been modified by depletion of tolerogenic compounds.

Embodiment 33. The composition of embodiment 32, wherein the tolerogenic compounds comprise Wul ligands.

Embodiment 34. The composition of embodiment 32 or 33, wherein the cancer cells have been modified by depletion of tolerogenic compounds by exposure to beta-methyl-cyclodextrin.

Embodiment 35. The composition of any one of embodiments 1-13, wherein the cancer cells have been modified to increase production of damage-associated molecular patterns (DAMP).

Embodiment 36. The composition of embodiment 35, wherein the cancer cells have been modified by exposure to gentamicin.

Embodiment 37. The composition of any one of embodiments 1-36, wherein the composition is free of viable cancer cells.

Embodiment 38. The composition of embodiment 37 for use in treating the patient's cancer.

Embodiment 39. A personalized immunotherapeutic product comprising the composition of embodiment 37.

Embodiment 40. The use of the composition of embodiment 37 or the personalized immunotherapeutic product of embodiment 39 in the treatment of the patient's cancer.

Embodiment 41. The method of treating cancer comprising administering the composition of embodiment 37, or the personalized immunotherapeutic product of embodiment 39, to the patient.

Embodiment 42. A method of making a personalized immunotherapeutic product against cancer for an individual cancer patient, comprising
obtaining tumor tissue from the patient;
obtaining blood from the patient; and
manipulating the tumor tissue and the blood to create a personalized immunotherapeutic product, wherein manipulating comprises ex vivo modification of cancer cells or DC obtained from the patient, or both, to improve the accumulation, or immunogenicity, of TAA expressed by the patient's cancer.

Embodiment 43. A method of making a personalized immunotherapeutic product against cancer tor an individual cancer patient, comprising:
obtaining tumor tissue from the patient;
obtaining blood from the patient;
manipulating the tumor tissue to enhance the accumulation of TAA; and
manipulating the blood to isolate monocytes and differentiate them into dendritic cells and, optionally, to enhance the antigen presentation ability of the dendritic cells;
to create a personalized immunotherapeutic product comprising the dendritic cells and cancer cell material from the manipulated tumor tissue.

Embodiment 44. The method of embodiment 42 or 43, wherein modification to improve the accumulation or immunogenicity of TAA comprises carrying out the modification referenced in any one of embodiments 1-36.

Embodiment 45. The method of any one of embodiments 42-44, wherein manipulating comprises inactivating the cancer cells.

Embodiment 46. The method of embodiment 45, wherein inactivating comprises exposure to gamma irradiation.

Embodiment 47. The method of embodiment 45, wherein inactivating comprises exposure to UV irradiation

Embodiment 48. The method of embodiment 45, wherein inactivating comprises exposure to X-ray irradiation.

Embodiment 49. The method of any one of embodiments 45-48, wherein inactivation comprises cell lysis.

Embodiment 50. The method of any one of embodiments 42-49, wherein obtaining comprises physically removing the tumor tissue and the blood from the patient.

Embodiment 51. The method of claim 50, further comprising sending the tumor tissue and the blood to a central facility having the capability to isolate cancer cells from the tumor tissue and to differentiate DC from monocytes in the blood, and wherein the central facility further has the capability to 1) modify the DC to increase cross-processing or 2) to modify the cancer cells to augment TAA content or enhance immunogenicity of the cancer cells or 3) both.

Embodiment 52. The method of any one of embodiments 42-49, wherein obtaining comprises receiving a shipment of the tumor tissue and the blood.

Embodiment 53. The method of any one of embodiments 42-52, further comprising isolating cancer cells from said tumor tissue.

Embodiment 54. The method of any one of embodiments 42-53, further comprising obtaining DC from said blood by differentiating monocytes.

Embodiment 55. The method of any one of embodiments 42-54, further comprising combining cancer cell material, comprising the cancer cells or a lysate thereof, with the DC in the presence of a DC maturation factor.

Embodiment 56. The method of embodiment 55 wherein the DC are immature DC at the time of combining.

Embodiment 57. The method of embodiments 55 or 56, wherein the DC maturation factor is lipopolysaccharide (LPS).

Embodiment 58. The method of any one of embodiments 42-57, further comprising modifying the DC to have an increased level of cross-presentation.

Embodiment 59. The method of any one of embodiments 42-58, further comprising modifying the cancer cells to increase production of DAMP.

Embodiment 60. The method of embodiments 58 or 59, wherein modifying comprises exposure of the DC or cancer cell to an aminoglycoside antibiotic.

Embodiment 61. The method of embodiment 60, wherein the aminoglycoside antibiotic comprises gentamicin.

Embodiment 62. The method of any one of embodiments 42-61, wherein modifying comprises exposure of the DC to a toll-like receptor 4 agonist.

Embodiment 63. The method of any one of embodiments 42-62, further comprising modifying the cancer cells to express or accumulate an increased amount of TAAs.

Embodiment 64. The method of embodiment 63, wherein the cancer cells are modified by exposure to a genome demethylation agent.

Embodiment 65. The method of embodiment 64, wherein the genome demethylation agent comprises decitabine.

Embodiment 66. The method of any one of embodiments 42-65, wherein the cancer cells are modified by exposure to a histone acetylation-promoting agent.

Embodiment 67. The method of embodiment 66, wherein the histone acetylation-promoting agent comprises a histone deacetylase inhibitor.

Embodiment 68. The method of embodiment 67, wherein the histone deacetylase inhibitor comprises valproic acid.

Embodiment 69. The method of any one of embodiments 42 68, wherein the cancer cells are modified by exposure to a proteasome inhibitor.

Embodiment 70. The method of embodiment 69, wherein the proteasome inhibitor comprises lactacystin, epoxomicin, beta-hydroxy-beta-methylbutyrate, or any combination thereof.

Embodiment 71. The method of any one of embodiments 42-70, wherein the cancer cells are modified by exposure to an E3 ligase inhibitor.

Embodiment 72. The method of any one of embodiments 42-71, wherein the cancer cells are modified by exposure to a PI3K/AKT/mTOR pathway activator.

Embodiment 73. The method of embodiment 72, wherein the PI3K/AKT/mTOR pathway activator comprises supranormal concentrations of leucine or arginine or both in the cell culture medium.

Embodiment 74. The method of embodiments 72 or 73, wherein the PI3K/AKT/mTOR pathway activator comprises a PTEN inhibitor.

Embodiment 75. The method of embodiment 74, wherein the PTEN inhibitor comprises bisperoxovanadium-1,10-phenanthroline (bpV(phen), bisperoxovanadium-5-hydroxypiridine-2-carboxyl (bpV(HOpic), bisperoxo-(bipyridine)-oxovanadate bpV(bipy), or any combination thereof.

Embodiment 76. The method of embodiments 74 or 75, wherein the PTEN inhibitor is used in conjunction with one or more hormones or growth factors.

Embodiment 77. The method of embodiment 76, wherein the one or more hormones or growth factors comprise insulin, thyroid hormone, basic FGF, EGF, or a combination thereof.

Embodiment 78. The method of any one of embodiments 42-77, wherein the cancer cells are modified by exposure to an inhibitor of apoptosis.

Embodiment 79. The method of embodiment 78, wherein the inhibitor of apoptosis is a caspase inhibitor.

Embodiment 80. The method of embodiment 79, wherein the caspase inhibitor comprises zVAD.fmk.

Embodiment 81. The method of any one of embodiments 42-80, wherein the cancer cells are modified by depletion of tolerogenic compounds.

Embodiment 82. The method of embodiment 81, wherein the tolerogenic compounds comprise Wnt ligands.

Embodiment 83. The method of embodiments 81 or 82, wherein the cancer cells are modified by depletion of tolerogenic compounds by exposure to beta-methyl-cyclodextrin.

Embodiment 84. The method of any one of embodiments 42-83, further comprising adding a cryoprotectant to the combined DC and cancer cell material 24-48 hours after they are combined and cryopreserving the combined material.

Embodiment 85. The method of treating cancer comprising administering the personalized immunotherapeutic product made by the method of any one of embodiments 42-84 to the individual cancer patient.

Embodiment 86. The use of the personalized immunotherapeutic product made by the method of any one of embodiments 42-84 in the treatment of cancer.

Embodiment 87. The use of the composition of any one of embodiments 1-37 in the manufacture of a medicament for the treatment of the patient's cancer.

Embodiment 88. The personalized immunotherapeutic product of embodiment 39, wherein the patient is human.

Embodiment 89. The personalized immunotherapeutic product of embodiment 88 containing 1 to 20×10⁶ DC per dose.

Embodiment 90. The method of treatment of embodiments 41 or 85, or the use of any one of embodiments 39 or 86, comprising administration by injection.

Embodiment 91. The method of treatment of embodiments 41 or 85, or the use of any one of embodiments 39 or 86, comprising administration by infusion.

Embodiment 92. The method of treatment of embodiments 41 or 85, or the use of any one of embodiments 39 or 86, further comprising administration of an immune checkpoint inhibitor.

Embodiment 93. The method or use of claim 92, where in the immune checkpoint inhibitor is an antibody specific for CTLA-4, PD-1, PD-L1, TIM-3, LAG-3, B7-H3, B7-H4, BTLA, ICOS, or OX40.

Embodiment 94. The method of treatment of embodiments 41 or 85, or the use of embodiments 39 or 86, comprising administration of a single dose.

Embodiment 95. The method of treatment of embodiments 41 or 85, or the use of embodiments 39 or 86, comprising administration at weekly intervals.

Embodiment 96. The method of treatment of embodiments 41 or 85, or the use of embodiments 39 or 86, comprising administration at monthly intervals.

Embodiment 97. The method of treatment of embodiments 41 or 85, or the use of embodiments 39 or 86, wherein the cancer is a carcinoma.

Embodiment 98. The method of treatment of embodiments 41 or 85, or the use of embodiments 39 or 88, wherein the cancer is a sarcoma.

Embodiment 99. The method of treatment of embodiments 41 or 85, or the use of embodiments 39 or 86, wherein the cancer is a leukemia or lymphoma.

Embodiment 100. The method of treatment of embodiments 41 or 85, or the use of embodiments 39 or 86, wherein the cancer is a cancer of the brain head & neck esophagus, lung, liver, pancreas, kidney, stomach, colon, prostate, breast, uterus, cervix, ovary, skin, bone, hematologic tissue, eye, or retina.

Embodiment 101. The method of treatment of embodiments 41 or 85, or the use of embodiments 39 or 86, wherein the cancer is melanoma, non-small cell lung cancer, glioblastoma, renal cell carcinoma, or colorectal cancer.

### EXAMPLES

The following non-limiting examples are provided for illustrative purposes only in order to facilitate a more complete understanding of representative embodiments now contemplated. These examples should not be construed to limit any of the embodiments described in the present specification, though they can support specific limitations found in the claims.

### Example 1

### Isolation of Live Cancer Cells from Tumors

In a typical preparation, tumor pieces obtained by surgical removal are dissected from normal tissue, mechanically minced to 2-3 mm diameter fragments and dissociated with an enzyme in cell culture media. The minced tumor pieces are subjected to continuous agitation in the presence of trypsin of collagenase for 0.5 to 3 hours at 37° C. Alternatively, dipase is used in lower concentration overnight or up to 72 hours refrigerated (˜4° C.), at room temperature (˜25° C.), or at 37° C. Digested extracellular matrix and other debris are removed by repeated cycles of centrifugation and resuspension. The live cancer cells are transferred into cell culture vessels and expanded in a nutriment rich media.

### Example 2

### Increased Protein Expression by Inhibition of Histone Deacetylases

Isolated live cancer cells are placed in tissue culture. During an augmentation phase, the tissue culture medium is supplemented with valproic acid or phenylbutyrate, or both, each at a concentration of 0.01 mM to 10 mM each. The level of histone acetylation, mRNA transcription, and protein expression, including expression of TAA, all increase.

### Example 3

### Increased Protein Expression by Inhibition DNA Methyltransferase

Isolated live cancer cells are placed in tissue culture. The tissue culture medium is supplemented with decitabine from the beginning of the augmentation phase for not less than one hour and up to the entire augmentation phase depending on concentration. A concentration of 100-500 nM can be used throughout the augmentation phase of cultivation. Alternatively a higher concentration of 1 μM-10 μM can be used at beginning of the augmentation phase and then removed or reduced to the lower concentration. Hypermethylation is reversed and expression of silenced germ line and tumor-specific antigens is increased and becomes more uniform throughout the cancer cell population.

### Example 4

### Increased Protein Content Due to Proteasome Inhibition

Isolated live cancer cells are placed in tissue culture. The tissue culture medium is supplemented with lactacystin at a concentration of 0.1-1 μM, epoxomicin at a concentration of 1-2 μM, and/or HMB at a concentration of 10-150 μg/mL. The inhibitor can be present throughout the augmentation phase of culture, or a portion thereof, but at least the last 24 hours prior to harvest. Doses in the lower end of the stated ranges are appropriate for longer term exposure. Doses at the high end of the state ranges can be used in the last 24 hours of cultivation. The content of protein (including normal, mutated and misfolded proteins) in the cancer cells is increased.

### Example 5

### Increased Cell Proliferation and Protein Production by Inhibition of PTEN

Isolated live cancer cells are placed in tissue culture in medium supplemented with leucine and arginine at a ratio of arginine:alanine of 70:1 and a ratio of leucine:alanine of 25:1. The tissue culture medium is further supplemented with bpV(phen), bpV(HOpic), or bpV(bipy) at a concentration of 5-20 μM. It is applied at least for 24 hours at the cell culture initiation and optionally continued during the entire cell culture period at a lower concentration. Throughout the period of PTEN inhibition, the culture medium is further supplemented with insulin, thyroid hormone, basic FGF and EGF.

### Example 6

### Rapid Cancer Cell Expansion and Accumulation of TAAs through Inhibition of Apoptosis

Isolated live cancer cells are placed in tissue culture. The tissue culture medium is supplemented with the broad spectrum caspase inhibitor Z-VAD-fmk at a concentration of 5-50 μM. The caspase inhibitor is added at the initiation of cell culture and maintained throughout the proliferation and augmentation phases of culture. The cultured cancer cells, proliferate, maintaining a high degree of viability and proteins, including TAA, accumulate to higher levels than in untreated cultures.

### Example 7

### Increasing Immunogenicity by Depletion of Wnt Signaling Ligands

Isolated live cancer cells are placed in tissue culture. One half to 3 hours prior to the end of the augmentation phase and preparation for DC loading, the tissue culture medium is supplemented with beta-methyl-cyclodextrin at a concentration of 0.5-20 mM. Cholesterol and Wnt ligands are depleted from the plasma membrane of the cancer cells.

### Example 8

### Harvest and Inactivation of Cancer Cells after Augmentation Culture

At the completion of the augmentation phase of culture, cancer cells are dissociated by trypsinization. The collected cells are washed free of culture medium and trypsin solution by 3 cycles of centrifugation in phosphate buffered saline. The cells are then irradiated for a total dose of 100Gy and lysed using 3 to 5 freeze/thaw cycles in cryoprotectant free media. Total protein is determined by the biuret method.

### Example 9

### Promotion of Cross-Processing in DC by treatment with an Aminoglycoside Antibiotic

PBMC are plated in culture medium and the monocytes allowed time to adhere, after which the non-adherent cells are washed away. Fresh medium supplemented with GM-CSF, IL-4 and 5-10 μ/ml gentamicin is added and the cells incubated for 3-5 days. The inactivated cancer cells or cell lysate, and LPS, are added and the gentamicin concentration is increased to 50-150 μ/ml and the DC are incubated another 24-48 hours.

### Example 10

### In vitro Application of a Proteasome Inhibitor and a Caspase Inhibitor To Increase Protein Accumulation in Tumor Cells

To benefit from the increased antigen content that can be obtained through use of a proteasome inhibitor, but also avoid triggering apoptosis, the proteasome inhibitor can be used in conjunction with a caspase inhibitor. First, to find the concentration of the proteasome inhibitor that caused minimal cell death, a range of bortezomib dilutions were tested in vitro on an established ovarian tumor line after reaching 70-90% confluence. The cultures were maintained in a standard DMEM:F12 media with 5% FBS. Bortezomib was reconstituted in DMSO and added directly to the cultures in concentrations from 0.1 to 100 nM

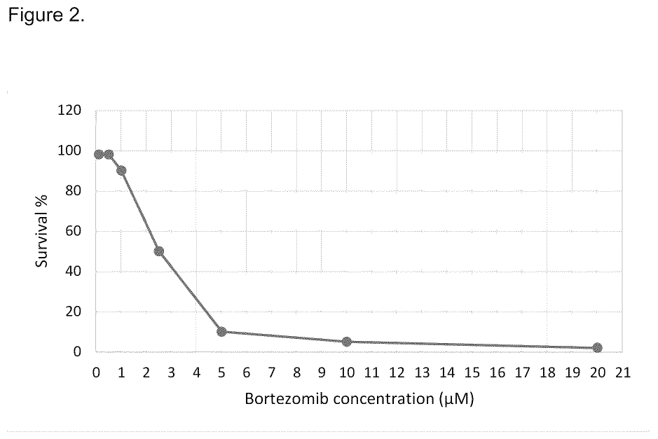

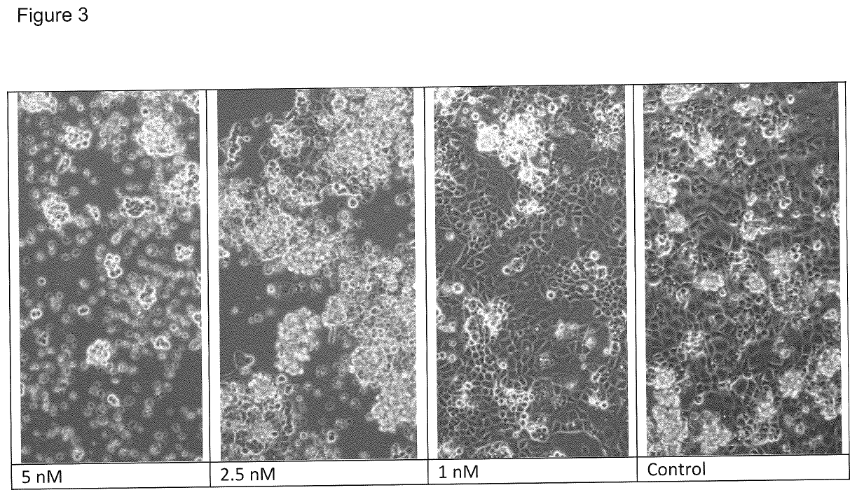

At concentrations above 5 nM bortezomib, all cells died within 24 hours. From 5 nM to 1 nM bortezomib, cell survived proportional with decreasing concentration. Below 1 nM concentration, bortezomib did not have effect on survival (FIGS. 2 and 3).

Next, the caspase inhibitor Z-VAD-fmk was tested at a concentration range of 10-100 μM in the same cell culture system and it was found that the caspase inhibitor did not have an effect on cell survival without an apoptotic challenge. A concentration of 20 μM was chosen for further experiments.

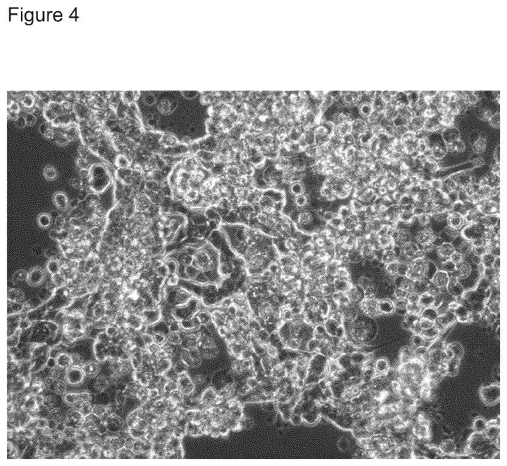

Then the sequential application of the caspase inhibitor and proteasome inhibitor was tested. The tumor cells were cultured in the presence of 20 μM caspase inhibitor Z-VAD-fmk for 24 hours and then the medium was exchanged for medium containing bortezomib and the cells culture for another 48 hours. Bortezomib was used at the lowest concentration of that induced cell death, 5 nM. We observed a cell survival of about 75% with a distinctive cell morphology, consisting in multinucleated, enlarged cell bodies, as exemplified in FIG. 4.

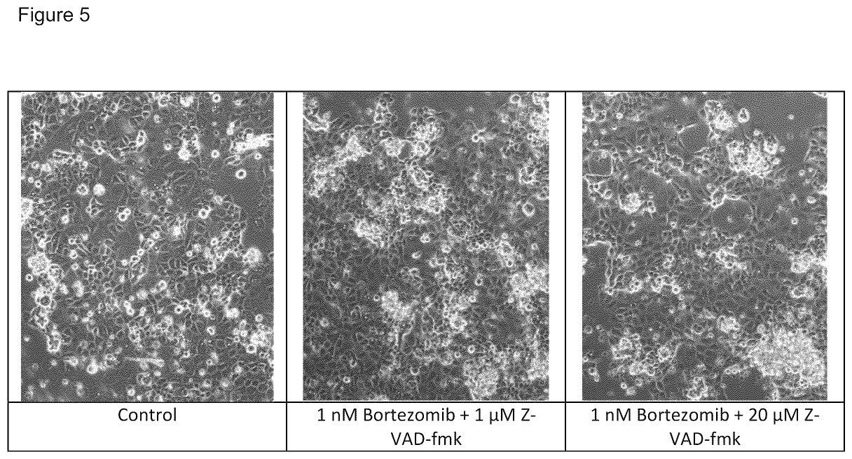

The simultaneous application of Bortezomib and Z-VAD-fmk was also tested using 1 nM Botezomib with either 1 or 20 μM Z-VAD-fmk. These treatments do not cause apparent changes in cell morphology or survival (see FIG. 5).

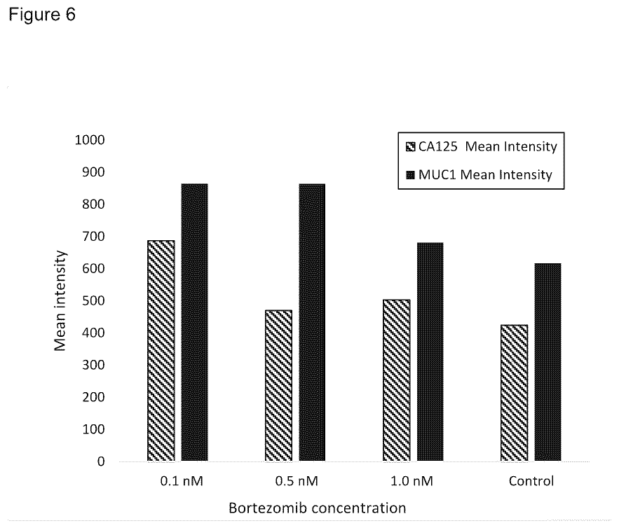

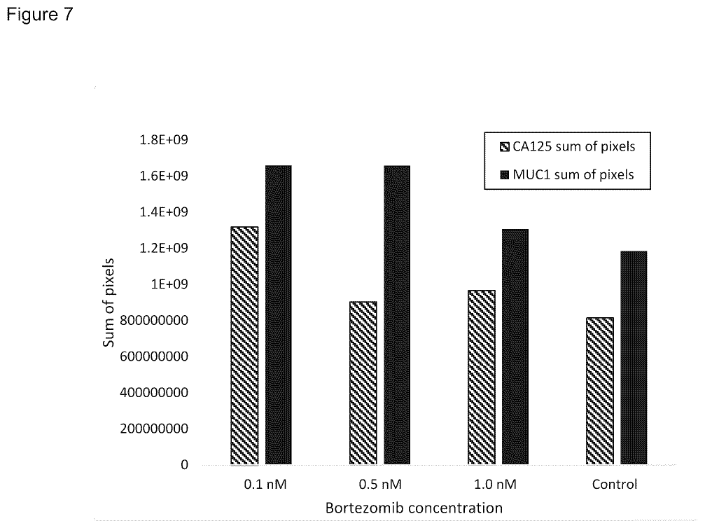

To evaluate protein content, we queried two targets that are commonly found in cancers: CA125, which is typically specific for ovarian cancer, and MUC1, which is common in many types of cancer (for example, colon, breast, ovarian, lung and pancreatic). The proteins were labeled using antibodies conjugated with Alexa Fluor 488 (green; anti-MUC1) or 594 (red; anti-CA125). The cells were imaged with an epifluorescence microscope (Nikon) with pre-set parameters for exposure and magnification. Each channel was analyzed for the maximum pixel intensity (demonstrating similar imaging parameters), the mean intensity demonstrating the increased or decreased target protein abundance, and the sum of labeled pixels proportional with the labeled target protein content, using the Nikon NIS-Elements software. As can be seen in FIGS. 6 and 7, bortezomib in concentrations of 0.1 nM to 1.0 nM (without caspase inhibitor) increase the abundance and content of these antigens in the treated tumor cells.

In addition, labeling for Ki67 was used to analyze differences in the proliferative status of the tumor cells under the various conditions. The Ki67 labeling did not show any differences in proliferative status.

These data demonstrate that increasing the protein content of tumor cells is possible in vitro by exposing the cells to a proteasome inhibitor at low concentration and optional in combination with a caspase inhibitor, without affecting cell viability or proliferative capacity. The treatment results in increased protein content and subsequently in neoantigen availability for antigen presenting cells. At the same time, the in vitro treatment of tumor cells prior exposure to dendritic cells, avoids the detrimental exposure in vivo of antigen presenting cells to proteasome inhibitors. These findings are important with immediate applicability to dendritic cell based immune therapies.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A composition for treating a disease, condition, or disorder, comprising:
a. cancer cells from a patient, wherein the cancer cells have been supplemented with a compound comprising cyclodextrin, wherein the cancer cells are depleted of (i) lipids, (ii) Wingless (Wnt) proteins, (iii) or cholesterol; and
b. dendritic cells from the patient, wherein the dendritic cells are modified by incubation with an aminoglycoside antibiotic.

2. (canceled)

3. A method of preparing a composition for treating a disease, condition, or disorder, comprising:
a. depleting cancer cells of a patient of lipids, cholesterol, or Wingless (Wnt) proteins, wherein the depleting comprises supplementing the cancer cells from a patient with a compound comprising cyclodextrin;
b. modifying dendritic cells of the patient to have increased cross presentation, wherein the modifying comprises incubating the dendritic cells with an aminoglycoside antibiotic;
c. inactivating and or lysing the cancer cells; and
d. adding the inactivated and or lysed cancer cells to the modified dendritic cells.

4. (canceled)

5. A method of treating a disease, condition, or disorder, comprising administering a composition, wherein the composition comprises:
a. cancer cells from a patient, wherein the cancer cells have been supplemented with a compound comprising cyclodextrin, and wherein the cancer cells are depleted of lipids, Wingless (Wnt) proteins or cholesterol; and
b. dendritic cells from the patient modified by an aminoglycoside antibiotic.

6. (canceled)

7. The method of claim 2 or 4, further comprising further incubating the dendritic cells after the cancer cells have been added.

8. The method of claim 2 or 5, wherein the aminoglycoside antibiotic comprises gentamicin.

9. The method of claim 2 or 5, wherein the lipids comprise cholesterol.

10. The composition of claim 1, wherein the cyclodextrin comprises beta-methyl-cyclodextrin.

11. The composition of claim 1, wherein the cancer cells are supplemented with the compound comprising cyclodextrin at a concentration of 0.5 to 20 mM.

12. The composition of claim 1, wherein the cancer cells are not viable.

13. The composition of claim 1, wherein administering the composition inhibits apoptosis of the cancer cells.

14. The method of claim 2 or 4, further comprising isolating the dendritic cells and/or the cancer cells.

15. The composition of claim 1, wherein the dendritic cells have been modified by exposure to a toll-like receptor 4 agonist.

16. The composition of claim 1, wherein the cancer cells express or accumulate an increased amount of TAAs.

17. The method of claim 2 or 4, further comprising exposing the cancer cells to a genome demethylation agent or a histone acetylation promoting agent.

18. The method of claim 2 or 4, further comprising exposing the cancer cells to a proteasome inhibitor and/or a caspase inhibitor.

19. The method of claim 2 or 4, further comprising exposing the cancer cells to an E3 ligase inhibitor.

20. The method of claim 2 or 4, further comprising exposing the cancer cells to a PI3K/AKT/mTOR pathway activator.

21. The composition of claim 1, wherein the cancer cells and are depleted of Wingless (Wnt) proteins and cholesterol.

22. The method of claim 3, wherein the cancer cells and are depleted of Wingless (Wnt) proteins and cholesterol.

23. The method of claim 5, wherein the cancer cells and are depleted of Wingless (Wnt) proteins and cholesterol.
